(19) United States
(12) Patent Application Publication
(10) Pub. No.: US 2024/0058407 A1
(43) Pub. Date: Feb. 22, 2024

(54) COMPOSITION FOR IMPROVING INTESTINAL MICROBIAL FLORA COMPRISING GINSENG EXTRACT OR FRACTION THEREOF

(71) Applicant: KOREA GINSENG CORP., Daejeon (KR)

(72) Inventors: Joon Hyuk HOU, Daejeon (KR); Hye Sun CHO, Daejeon (KR); Seung Ho LEE, Daejeon (KR)

(73) Assignee: KOREA GINSENG CORP., Daejeon (KR)

(21) Appl. No.: 18/258,820

(22) PCT Filed: Aug. 24, 2021
(86) PCT No.: PCT/KR2021/011306

§ 371 Date: Jun. 22, 2023

(30) Foreign Application Priority Data
Dec. 24, 2020 (KR) 10-2020-0183829

(51) Int. Cl. A61K 36/258; A61K 31/19; A61P 1/00; A61K 36/481; A61K 36/484; A61K 36/539; A61K 36/725

(52) U.S. Cl. CPC A61K 36/258; A61K 31/19; A61P 1/00; A61K 36/481; A61K 36/484; A61K 36/539; A61K 36/725; A61K 2236/333; A61K 2236/51

(57) ABSTRACT

The present invention provides a composition for improving intestinal microbial flora, the composition comprising: a ginseng extract or a fraction thereof; and sodium propionate. The composition for improving intestinal microbial flora of the present invention is capable of increasing the proportion of beneficial bacteria in intestinal microbial flora by promoting the growth of beneficial bacteria while inhibiting the growth of harmful bacteria, and accordingly, the composition of intestinal microbial flora can be improved and the balance thereof maintained, ultimately assisting in improving the health of the body, such as by strengthening immunity and improving skin texture.

### TECHNICAL FIELD

The present invention relates to a composition for improving intestinal microbial flora comprising a ginseng extract or a fraction thereof.

### BACKGROUND ART

The microbiome is a compound word created by combining microbiota and genome, and may be referred to as a microbial community that includes microorganisms that inhabit or coexist in all environments, such as humans, animals and plants, soil, sea, lakes, rocks, and air, and entire genetic information thereof. Recently, the microbiome is also referred to as the microbial community in various environments. Various studies on microbiome, such as plant microbiome, animal microbiome, environmental microbiome, human microbiome, intestinal microbiome, and skin microbiome have been conducted, and among them, most research on the human microbiome has been conducted.

The human body is a complex and life-active ecosystem, and various microorganisms exist in the stomach, the skin, and the like of the human body. In the stomach, the skin, and the like, 500 to 1000 or more different microorganisms coexist and constitute microbial flora. Intestinal microorganisms existing in the stomach serve to synthesize materials such as vitamins to supply the materials to the human body, and to inhibit infection by pathogenic microorganisms introduced through contaminated food.

The intestinal microorganisms are transplanted from the mother from birth and influenced by the environment to form different intestinal microbial flora for each individual, and innumerable numbers of microbial flora live together in the body and are in balance to take charge of various physiological functions. Recently, since the intestinal microbial flora of obese and normal people varies, it has been found that a composition of a specific microorganism may affect the body weight. When the intestinal microbial flora was transplanted into another intestine, a change in body weight was induced, so that it was also found that the obesity was closely associated with the intestinal microorganisms.

Prebiotics are indigestible food additives that have beneficial effects on a host by restricting the growth of intestinal harmful microorganisms or selectively promoting the growth of beneficial microorganisms. As the prebiotics, oligosaccharides (fructooligosaccharide, galactooligosaccharide, isomaltooligosaccharide, xylooligosaccharide, and chitin oligosaccharide), dietary fiber (dextrin and inulin), and the like are known. Recently, the importance of prebiotics that may help in intestinal health by acting as food for intestinal beneficial bacteria has been more highlighted, and research on new materials that may be used as prebiotics has been continuously conducted (Patent Document 1).

Ginseng (Panax ginseng C.A. Meyer) is a perennial herb belonging to the genus Panax of the Araliaceae family, and one of the herbal medicines used in oriental medicine, and has a sweet and bitter taste, has warm energy, and has herbal effects such as strengthening the vitality, protecting the spleen, and strengthening the lungs.

The main physiologically active materials of the ginseng include saponin, also called ginsenoside, an essential oil ingredient, polyacetylene, a phenol ingredient, glycoside, acidic peptide, and the like, and include additionally various ingredients such as vitamins, sugars, and minerals. The general physiologically active efficacy of the ginseng has been reported to have an effect on the central nervous system, an effect of enhancing immune functions, an anticancer effect, and the like. In particular, about 40 types of ginsenosides have been discovered so far, and have been confirmed to widely affect the endocrine system, the immune system, the metabolic system, and the like including the central nervous system to exhibit excellent effects on the regulation of body functions, that is, normalization of physiological functions. These ginsenosides have similar or opposite effects, and have been known to exhibit various effects by a specific ingredient alone or through interaction of several types thereof.

The present inventors found that a red ginseng extract exhibited an excellent effect in promoting the growth of intestinal beneficial bacteria and discovered a possibility that the red ginseng extract can be used as prebiotics, but the red ginseng extract has a problem of promoting the growth of harmful bacteria as well as the intestinal beneficial bacteria. Accordingly, while research on a prebiotic composition capable of inhibiting the growth of harmful bacteria and selectively promoting only beneficial bacteria has continued, the present inventors confirmed that when sodium propionate was mixed with the red ginseng extract, the growth of harmful bacteria was effectively inhibited while the growth of beneficial bacteria was promoted, and then completed the present invention.

### DISCLOSURE

### Technical Problem

An object of the present invention is to provide a composition for improving intestinal microbial flora capable of inhibiting the growth of harmful bacteria while promoting the growth of beneficial bacteria in the intestinal microbial flora.

### Technical Solution

An aspect of the present invention provides a composition for improving intestinal microbial flora comprising a ginseng extract or a fraction thereof and sodium propionate.

Another aspect of the present invention provides a pharmaceutical composition for preventing, improving or treating intestinal inflammatory diseases or intestinal motility disorders comprising a ginseng extract or a fraction thereof and sodium propionate.

### Advantageous Effects

According to the present invention, the composition for improving the intestinal microbial flora may increase the proportion of beneficial bacteria in the intestinal microbial flora by inhibiting the growth of harmful bacteria while promoting the growth of beneficial bacteria. Accordingly, the composition of the present invention can improve the configuration of the intestinal microbial flora and maintain the balance thereof, and ultimately assist in improving the health of the body, such as strengthening immunity, improving skin texture, and the like.

### DESCRIPTION OF DRAWINGS

FIG. 1 illustrates growth promoting effects on beneficial bacteria and harmful bacteria, when a red ginseng purified water extract of Preparation Example 1, a mixture of red ginseng extract/fraction of Preparation Example 3, and fructooligosaccharide are treated by concentration, respectively.

### BEST MODE

Hereinafter, the present invention will be described in detail.

The terms and words used in the present specification and claims should not be construed as limited to ordinary or dictionary meanings, and should be construed in a sense and concept consistent with the technical idea of the present invention, based on the principle that the inventor can properly define the concept of a term to describe his invention in the best way possible.

In the present invention, ‘microbial flora’ refers to a microbial community that includes all of bacteria, fungi and other prokaryotes existing in the human body, and mainly inhabits the stomach, the skin, and the like.

In the present invention, ‘intestinal microbial flora’ means a microbial community existing in the intestine.

An aspect of the present invention provides a composition for improving intestinal microbial flora comprising a ginseng extract or a fraction thereof; and sodium propionate.

In the present invention, ‘improving the intestinal microbial flora’ refers to promoting the growth of intestinal beneficial bacteria, inhibiting the growth of intestinal harmful bacteria, or maintaining the balance of intestinal beneficial bacteria and harmful bacteria.

In the present invention, ‘prebiotics’ as a food additive that is not digested well in the body is a material capable of stimulating the growth or activity of specific beneficial bacteria without hydrolysis or absorption in the upper part of the gastrointestinal tract to induce the intestinal microbial flora in a direction beneficial to the health of a host. In the present invention, a ‘prebiotic composition’ may be used in the same meaning as the composition for improving the intestinal microbial flora.

In the present invention, the ‘intestinal beneficial bacteria’ refers to existing beneficial bacteria that live in the intestine and bacteria that have a beneficial effect on an intestinal environment when being ingested to reach the intestine. The intestinal beneficial bacteria may be, but are not particularly limited thereto, Bifidobacterium, Lactobacillus, Lactococcus, Streptococcus, Leuconostoc, Akkermansia, Faecalibacterium, Enterococcus, Bacillus, and the like.

Specifically, the intestinal beneficial bacteria may include at least one selected from the group consisting of L. acidophilus, L. casei, L. gasseri, L. delbrueckii subsp. Bulgaricus, L. helveticus, L. fermentum, L. paracasei, L. plantarum, L. reuteri, L. rhamnosus, L. salivarius, L. delbrueckii subsp. lactis, L. lactis, L. lactis subsp. cremoris, L. lactis subsp. lactis biovar diacetylactis, L. lactis subsp. lactis, E. faecium, E. faecalis, S. thermophilus, L. mesenteroides, L. pseudomesenteroides, B. bifidum, B. infantis, B. breve, B. longum, B. animalis subsp. lactis, B. lactis, and B. coagulans.

In the present invention, the ‘intestinal harmful bacteria’ refer to existing harmful bacteria that live in the intestine and bacteria that have a harmful effect on an intestinal environment when being ingested to reach the intestine. The intestinal harmful bacteria may be, but are not particularly limited thereto, Escherichia, Fusobacterium, Clostridium, Staphylococcus, or Porphyromonas, Bacteroides, Vibrio, Salmonella, Shigella, Akkermansia, Eubacterium, Pseudomonas, Candida, Bacteroides, and the like.

Specifically, the intestinal harmful bacteria may include at least one selected from the group consisting of E. coli, S. aureus, Acidaminobacter, Enterobacteriaceae, A. mucimphila, E. rectale, C. coccoides, C. lituseburense, C. histolyticum, C. perfringens, C. difficile, Veillonellaceae, Proteobacteria, Betaproteobacteria, P. aeruginosa, C. albicans, and B. vulgatus.

In the present invention, the ‘extract’ refers to a material extracted from a raw material by any method, and is used in the meaning of including, without limitation, an extract extracted above, a concentrate obtainable therefrom, and dry matter and powder of the concentrate.

The extract may be obtained by extraction from a raw material or a dried matter thereof, and the raw material of the extract may be used without limitation, such as cultivated or commercially available materials.

In the present invention, when the ‘ginseng extract’ is extracted and obtained from ginseng, as an extraction method, all known conventional extraction methods such as solvent extraction, ultrasonic extraction, filtration, and reflux extraction may be used, and the ginseng extract may be prepared, preferably, using solvent extraction or reflux extraction. The extraction process may be repeated several times, and thereafter, steps such as concentration or freeze-drying may be additionally performed. Specifically, the obtained extract is concentrated under reduced pressure to obtain a concentrate, and after freeze-drying the concentrate, high-concentration extract powder may be prepared using a grinder. The extract also includes a fraction obtained by further fractionating the extract.

The ginseng extract may be extracted using water, an organic solvent, or a mixture thereof as an extraction solvent. The organic solvent may be any one selected from the group consisting of alcohol, preferably C1-C4 lower alcohol, hexane (n-hexane), ether, glycerol, propylene glycol, butylene glycol, ethyl acetate, methyl acetate, dichloromethane, chloroform, ethyl acetate, benzene, and mixed solvents thereof, preferably ethanol. For example, when a mixture of water and the organic solvent is used as the extraction solvent, the mixture of water and the organic solvent may be preferably a mixture of water and C1-C4 lower alcohol, more preferably a mixture of water and ethanol.

The mixture of water and ethanol may be 3% (v/v) or more and less than 100% (v/v) of an aqueous ethanol solution, for example, 5% (v/v) to 99% (v/v) of an aqueous ethanol solution, preferably 10% (v/v) to 95% (v/v) of an aqueous ethanol solution, more preferably 15% (v/v) to 90% (v/v) of an aqueous ethanol solution, and even more preferably 20% (v/v) to 80% (v/v) of an aqueous ethanol solution, but is not limited thereto.

When the ginseng extract is prepared, the extraction may be performed at 10° C. to 100° C. or 50° C. to 100° C., preferably 60° C. to 90° C., and more preferably 70° C. to 80° C., but is not limited thereto. The extraction may be performed for 2 hours to 12 hours, preferably for 5 hours to 10 hours, and more preferably for 6 hours to 8 hours. The extraction may be performed 1 to 7 times, preferably 1 to 4 times, and more preferably 1 to 2 times, but is not limited thereto.

A filler, such as silica gel, activated alumina, synthetic polymer, magnesium silicate, activated carbon, cellulose, and ion exchange resins, may be used in a column used for preparing the purified fraction. An aromatic synthetic resin is preferably used as the filler, that is, the column may be an aromatic-type synthetic adsorbent column, and a Diaion HP-20 synthetic adsorbent is more preferably used as the filler, but the present invention is not limited thereto. Separation using the column may be performed once to several times until a fraction having desired purity is purified, and concentration and recrystallization may be performed as necessary.

When preparing the purified fraction, an elution solvent may be any one selected from the group consisting of water, an organic solvent, and a mixture thereof. The organic solvent may be any one selected from the group consisting of alcohol, hexane (n-hexane), ether, glycerol, propylene glycol, butylene glycol, ethyl acetate, methyl acetate, dichloromethane, chloroform, ethyl acetate, benzene, acetone, acetonitrile, and mixed solvents thereof, preferably, the organic solvent may be any one selected from the group consisting of acetone, acetonitrile, C₁ to C₄ alcohol, and mixed solvents thereof, and the organic solvent may be at least one of ethanol or methanol, and more preferably ethanol, but is not limited thereto.

When the elution solvent is ethanol in the preparation of the purified fraction, the elution solvent may be ethanol or an aqueous ethanol solution, and the aqueous ethanol solution may be, for example, 30% (v/v) to 100% (v/v) of an aqueous ethanol solution, 50% (v/v) to 100% (v/v) of an aqueous ethanol solution, or 70% (v/v) to 100% (v/v) of an aqueous ethanol solution, but is not particularly limited thereto. When the concentration of the aqueous ethanol solution used as the elution solvent is less than the lower limit, the activity of the fraction obtained by adding the obtained fraction may not be sufficient.

When preparing the purified fraction, the adding of the elution solvent includes flowing the elution solvent to the column, for example, flowing the elution solvent under pressure. Specifically, the column purification fraction may be performed by including: 1) passing the ginseng extract through a column; and 2) adding an aqueous ethanol solution to the column through which the ginseng extract has passed to obtain a fraction.

The composition for improving the intestinal microbial flora of the present invention may use a purified fraction obtained by purifying the ginseng extract through the column in addition to the ginseng extract, in order to increase the effect of promoting the growth of intestinal beneficial bacteria or inhibiting the growth of intestinal harmful bacteria. In other words, in the present invention, the ginseng extract or the fraction thereof may be used to refer to including both the ginseng extract and the fraction of the ginseng extract.

In one embodiment, the composition for improving the intestinal microbial flora of the present invention may include a ginseng extract; a fraction of the ginseng extract; and sodium propionate. For the specific details regarding the fraction, the same contents as described above for the purified fraction may be applied.

The composition for improving the intestinal microbial flora may include the ginseng extract and the fraction of the ginseng extract at a weight ratio of 1:0.01 to 1:1, 1:0.05 to 1:0.5, or 1:0.07 to 1:0.3. Preferably, the composition for improving the intestinal microbial flora may include the ginseng extract and the fraction of the ginseng extract at a weight ratio of about 1:0.1. When the numerical range is satisfied, the composition for improving the intestinal microbial flora may increase the effect of promoting the growth of intestinal beneficial bacteria, or inhibiting the growth of intestinal harmful bacteria.

In the composition for improving the intestinal microbial flora of the present invention, the ginseng extract or the fraction thereof may be included at a concentration of 0.1 to 100 mg/ml, 0.5 to 50 mg/ml, or 1 to 25 mg/ml, particularly preferably at a concentration of 1.25 to 10 mg/ml.

In the present invention, the ‘sodium propionate’ is a white crystalline solid as a sodium salt of propionic acid, and is deliquescent in humid air.

The composition for improving the intestinal microbial flora of the present invention may include the ginseng extract or the fraction thereof and the sodium propionate at a weight ratio of approximately 8:1 to 1:8, 7:1 to 1:7, 6:1 to 1:6, 5:1 to 1:5, 4:1 to 1:4, 3:1 to 1:3, or 2:1 to 1:2. When the numerical range is satisfied, the composition may be excellent in the effect of promoting the growth of intestinal beneficial bacteria or inhibiting the growth of intestinal harmful bacteria.

In addition, the composition for improving the intestinal microbial flora of the present invention may further include a plant extract or a fraction thereof. The plant extract may include a scutellaria extract, a licorice extract, a jujube extract and an Astragali Radix extract.

When each extract included in the plant extract is prepared, all known conventional extraction methods such as solvent extraction, ultrasonic extraction, filtration, and reflux extraction may be used as the extraction method, and each extract may be prepared, preferably, using solvent extraction or reflux extraction. The extraction process may be repeated several times, and thereafter, steps such as concentration or freeze-drying may be additionally performed. Specifically, the obtained extract is concentrated under reduced pressure to obtain a concentrate, and after freeze-drying the concentrate, high-concentration extract powder may be prepared using a grinder.

When each extract included in the plant extract is prepared, the extract may be extracted using water, an organic solvent, or a mixture thereof as an extraction solvent. The organic solvent may be any one selected from the group consisting of alcohol, preferably C₁-C₄ alcohol, hexane (n-hexane), ether, glycerol, propylene glycol, butylene glycol, ethyl acetate, methyl acetate, dichloromethane, chloroform, ethyl acetate, benzene, and mixed solvents thereof, but is not limited thereto. Preferably, water may be used as the extraction solvent.

When preparing each extract, the extraction may be performed at 15° C. to 120° C., 20° C. to 32° C., 22° C. to 30° C., 23° C. to 27° C., 40° C. to 120° C., 45° C. to 110° C., 50° C. to 100° C., or 60° C. to 100° C., but is not limited thereto. The extraction may be performed for 2 hours to 24 hours, preferably for 4 hours to 18 hours, and more preferably for 6 hours to 16 hours. The extraction may be performed 1 to 7 times, preferably 1 to 4 times, and more preferably 1 to 2 times, but is not limited thereto.

In the plant extracts including the scutellaria extract, the licorice extract, the jujube extract and the Astragali Radix extract, as a mixing proportion of each plant extract with respect to the total weight of the mixed plant extract, the scutellaria extract may be included in 20 to 60 parts by weight, the licorice extract may be included in 10 to 50 parts by weight, the jujube extract may be included in 1 to 30 parts by weight, and the Astragali Radix extract may be included in 1 to 30 parts by weight, but the present invention is not limited thereto.

Preferably, the scutellaria extract may be included in 25 to 55 parts by weight, the licorice extract may be included in 15 to 45 parts by weight, the jujube extract may be included in 3 to 25 parts by weight, and the Astragali Radix extract may be included in 3 to 25 parts by weight. More preferably, the scutellaria extract may be included in 30 to 50 parts by weight, the licorice extract may be included in 20 to 40 parts by weight, the jujube extract may be included in 5 to 20 parts by weight, and the Astragali Radix extract may be included in 5 to 20 parts by weight. When each of the scutellaria extract, the licorice extract, the jujube extract and the Astragali Radix extract is included out of the range, the effect of promoting the growth of intestinal beneficial bacteria or inhibiting the growth of intestinal harmful bacteria may be reduced.

The composition for improving the intestinal microbial flora of the present invention may include the ginseng extract or the fraction thereof and the plant extract or the fraction thereof at a weight ratio of approximately 7:1 to 1:7, 6:1 to 1:6, 5:1 to 1:5, or 4:1 to 1:4. When the numerical range is satisfied, the composition may be excellent in the effect of promoting the growth of intestinal beneficial bacteria or inhibiting the growth of intestinal harmful bacteria.

The composition for improving the intestinal microbial flora of the present invention may be a health functional food composition or a food additive composition. When the composition for improving the intestinal microbial flora of the present invention is used as the health functional food composition or the food additive composition, within a range without impairing the effect of the present invention in addition to the ginseng extract or the fraction thereof and the sodium propionate of the present invention, preferably, the composition may further include other ingredients and the like capable of giving a synergistic effect to the effect of the ginseng extract or the fraction thereof and the sodium propionate. For example, the composition may include a conventional adjuvant, such as a stabilizer, a solubilizer, vitamins and the like, or a carrier.

In addition, the composition may include ingredients commonly added during food preparation, and includes, for example, proteins, carbohydrates, fats, nutrients, seasonings and flavoring agents. Examples of the carbohydrates may include monosaccharides, for example, glucose, fructose, and the like; disaccharides, for example, maltose, sucrose, oligosaccharide, and the like; and polysaccharides, for example, general sugars such as dextrin, cyclodextrin, and the like and sugar alcohols such as xylitol, sorbitol, erythritol, and the like. As the flavoring agent, a natural flavoring agent, taumatin, a stevia extract, and a synthetic flavoring agent may be used. For example, when the composition of the present invention is prepared as a drink, in addition to the ginseng extract or the fraction thereof and the sodium propionate of the present invention, citric acid, high fructose corn syrup, sugar, glucose, acetic acid, malic acid, fruit juice, natural extracts, etc. may be further included.

There is no particular limitation on a type of food using the health functional food composition or the food additive composition. Examples of food capable of adding the composition including the ginseng extract of the fraction thereof and the sodium propionate of the present invention include meat, sausage, bread, chocolate, candy, snacks, confectionery, pizza, ramen, other noodles, gums, dairy products including ice cream, various soups, beverages, tea, drinks, alcoholic beverages, vitamin complexes, and the like, and include all foods in the ordinary sense.

The food may be prepared and processed in the form of tablets, capsules, powders, granules, liquids, pills, and the like for the purpose for improving the intestinal microbial flora.

For example, the food in the form of tablets may produced by granulating a mixture obtained by mixing the health functional food composition or the food additive composition with an excipient, a binder, a disintegrant, and other additives according to a general method, and then compression-molding the mixture with a slip modifier and the like or directly. In addition, the food in the form of tablets may contain a sweetener or the like as needed.

A hard capsule agent among foods in the form of capsules may be prepared by filling a mixture of the health functional food composition or the food additive composition and additives such as an excipient in conventional hard capsules. A soft capsule agent may be prepared by filling a mixture of the health functional food composition or the food additive composition and additives such as an excipient in capsule bases such as gelatin. The soft capsule agent may contain a plasticizer such as glycerin or sorbitol, a colorant, a preservative, and the like if necessary.

The food in the form of pills may be prepared by molding a mixture of the health functional food composition or the food additive composition and an excipient, a binder, a disintegrant, and the like by conventional known methods, and may also be coated with white sugar or other coating agents or surface-coated with a material such as starch and talc, if necessary.

The food in the form of granules may be prepared by granulizing a mixture of the health functional food composition or the food additive composition and an excipient, a binder, a disintegrant, and the like by conventional known methods and may contain a flavoring agent, a sweetener, and the like, if necessary.

Another aspect of the present invention provides a pharmaceutical composition for preventing, improving or treating intestinal inflammatory diseases or intestinal motility disorders comprising a ginseng extract or a fraction thereof; and sodium propionate.

In the present invention, the ‘intestinal inflammatory diseases’ may be one or more selected from the group consisting of intestinal Behcet's disease, Crohn's disease, ulcerative enteritis, acute enteritis, and chronic enteritis.

In the present invention, the ‘intestinal motility disorders’ may be one or more selected from the group consisting of functional dyspepsia, irritable bowel syndrome, intestinal obstruction, constipation and diarrhea.

Within a range without impairing the effect of the present invention in addition to the ginseng extract or the fraction thereof and the sodium propionate of the present invention, preferably, the pharmaceutical composition of the present invention may further include other ingredients and the like capable of giving a synergistic effect to the effect of the ginseng extract or the fraction thereof and the sodium propionate. For example, the pharmaceutical composition of the present invention may include a conventional adjuvant, such as an antioxidant, a stabilizer, a solubilizer, vitamins and the like, or a carrier.

The route of administration of the pharmaceutical composition includes oral, intravenous, intramuscular, intraarterial, transdermal, subcutaneous, intraperitoneal, intranasal, enteral, topical, sublingual or rectal, and for example, a topical application method by application may be applied, but preferably, the pharmaceutical composition may be administered through the oral cavity.

The pharmaceutical composition of the present invention may be administered in a pharmaceutically effective dose. In the present invention, the “pharmaceutically effective dose” refers to an amount enough to treat diseases at a reasonable benefit/risk ratio applicable to medical treatment, and an effective dose level may be determined according to factors including a kind of subject, the severity, age, gender, a type of disease, the activity of a drug, sensitivity to a drug, a time of administration, a route of administration, an excretion rate, duration of treatment, and agents to be simultaneously used, and other factors well-known in the medical field. The composition of the present invention may be administered as an individual therapeutic agent or administered in combination with other therapeutic agents, and sequentially or simultaneously administered with conventional therapeutic agents. In addition, the pharmaceutical composition may be administered singly or in multiple. It is important to administer an amount capable of obtaining a maximum effect with a minimal amount without side effects in consideration with all the factors, and the amount thereof may be easily determined by those skilled in the art.

The pharmaceutical composition of the present invention is not particularly limited and applicable to any individual for the purpose of preventing, improving or treating intestinal inflammatory diseases or intestinal motility disorders. For example, the pharmaceutical composition of the present invention can be used not only for humans, but also for non-human animals such as monkeys, dogs, cats, rabbits, guinea pigs, rats, mice, cattle, sheep, pigs, goats, etc., birds and fish.

The pharmaceutical composition may contain one or more active ingredients having the same or similar function in addition to the ginseng extract or the fraction thereof and the sodium propionate. The pharmaceutical composition may be formulated and used in oral formulations, such as powders, granules, tablets, capsules, suspensions, emulsions, syrups, etc., according to a general method, respectively.

Solid formulations for oral administration include powders, granules, tablets, capsules, soft capsules, pills, and the like. Liquid formulations for oral administration may correspond to suspensions, oral liquids, emulsions, syrups, and the like, and may include various excipients, for example, a wetting agent, a sweetener, an aromatic agent, a preserving agent, and the like, in addition to water and liquid paraffin which are commonly used as simple diluents.

The pharmaceutical composition may additionally contain an adjuvant such as a preservative, a stabilizer, a hydrating agent or an emulsification accelerator, a salt and/or a buffer for controlling the osmotic pressure, and other therapeutically useful materials, and may be formulated according to a general method, such as mixing, granulating or coating.

The dose of the pharmaceutical composition may variously vary according to various factors including the age, weight, general health, and sex of a subject, an administration time, a route of administration, an excretion rate, a drug combination, and the severity of a specific disease.

In addition, the pharmaceutical composition of the present invention may be used alone or in combination with surgery, radiation therapy, hormone therapy, chemotherapy, and methods of using biological response modifiers. In addition, the pharmaceutical composition may be administered in a dose within the range of 0.001 to 200 mg/kg based on an adult, but the dose is not limited to the scope of the present invention.

Mode for Implementing the Invention Hereinafter, the present invention will be described in detail by Examples.

However, the following Examples are just illustrative of the present invention, and the contents of the present invention are not limited to Preparation Examples, Examples and Experimental Examples below.

### <Preparation Example 1> Preparation of Red Ginseng Purified Water Extract

1 kg of 6-year-old red ginseng was added with 6-fold purified water, extracted at room temperature for 8 hours, and then concentrated under reduced pressure to prepare a red ginseng concentrate (hereinafter referred to as a ‘red ginseng purified water extract’).

### <Preparation Example 2> Preparation of Ethanol Fraction of Red Ginseng Aqueous Ethanol Solution

1 kg of red ginseng was added with 5.5-fold 70% (v/v) ethanol and extracted once at 50° C. for 10 hours. The ethanol extract flowed through an HP-20 column to be adsorbed, and then the column was eluted with 70% ethanol. The eluate was freeze-dried to obtain 30 g or more of a powdery fraction (hereinafter referred to as an ‘ethanol fraction of the red ginseng aqueous ethanol solution’).

### <Preparation Example 3> Preparation of Mixture of Red Ginseng Extract/Fraction

The red ginseng purified water extract prepared in Preparation Example 1 and the ethanol fraction of the red ginseng aqueous ethanol solution prepared in Preparation Example 2 were mixed in a weight ratio of 10:1 to prepare a mixture of red ginseng extract/fraction.

### <Preparation Example 4> Preparation of Plant Extract

Scutellaria, licorice, Astragali Radix and jujube were washed 2 to 3 times with purified water, respectively, and then put into an extractor, and added with 5-fold purified water. After extracting at a temperature of 60° C. for 8 hours, centrifugation was performed to separate extraction residue, and then a filtrate was mixed with glycerin and concentrated to make 60 brix or more, to prepare a Scutellaria concentrate, a licorice concentrate, an Astragali Radix concentrate and a jujube concentrate.

38 g of the Scutellaria concentrate, 27 g of the licorice concentrate, 8.5 g of the jujube concentrate, 6.5 g of the Astragali Radix concentrate, and 20 g of purified water were mixed to obtain plant extracts.

### <Example 1> Confirmation of Effect of Promoting Growth of Intestinal Beneficial Bacteria and Harmful Bacteria

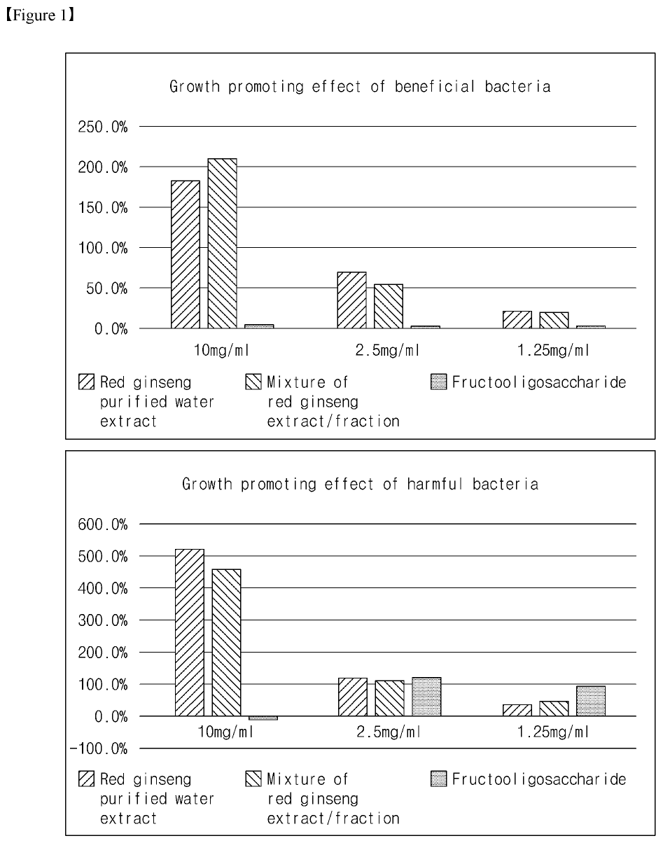

In order to confirm an effect of promoting the growth of intestinal beneficial bacteria and harmful bacteria, the red ginseng purified water extract of Preparation Example 1, the mixture of the red ginseng extract/fraction of Preparation Example 3, and fructooligosaccharide were dissolved in physiological saline at concentrations of 1.25, 2.5 and 10 mg/ml. S. aureus (Strain No. 39149, KCTC of Korea Research Institute of Bioscience and Biotechnology) and B. bifidum (Strain No. 3281, KCTC of Korea Research Institute of Bioscience and Biotechnology) were inoculated in the prepared sample at a concentration of 1×10⁶ cfu/ml and then cultured for 24 hours in a shaking incubator. After collecting and centrifuging the solution in which the strains were grown to remove a supernatant, the same amount of physiological saline was added to make a homogenate, and then an absorbance at 600 nm was measured, and increased amounts in the strains were illustrated in FIG. 1 compared with an untreated group.

From the result of FIG. 1, it can be confirmed that the red ginseng purified water extract has an excellent effect of promoting the growth of beneficial bacteria compared to fructooligosaccharide used as a conventional prebiotic ingredient. However, the red ginseng purified water extract of Preparation Example 1 exhibited a high effect of promoting the growth of not only beneficial bacteria but also harmful bacteria.

In addition, in the case of the mixture of red ginseng extract/fraction, the effect of selectively promoting only the growth of beneficial bacteria versus harmful bacteria was exhibited, but it was determined that the degree thereof was not sufficient. Therefore, the present inventors have confirmed the need to develop a composition capable of selectively selecting the growth of beneficial bacteria versus harmful bacteria.

### Example 2

2-1. Preparation of Composition for Improving Intestinal Microbial Flora

To 10 mg/ml of the mixture of red ginseng extract/fraction, 5 mg/ml of sodium propionate, 10 μl/ml of the plant extract of Preparation Example 4, 5 mg/ml of sodium benzoate, and 5 mg/ml of potassium sorbate were respectively mixed to prepare mixtures.

2-2. Confirmation of Selective Growth Promoting Effect of Beneficial Bacteria Versus Harmful Bacteria

In order to confirm whether each mixture selectively promoted the growth of beneficial bacteria versus harmful bacteria, each mixture was treated with physiological saline, and then the degrees of the growth of beneficial bacteria and harmful bacteria were measured in the same manner as in Example 1, and the increased amounts of the strains were calculated as a percentage compared to an untreated group and were shown in Table 1 below.

TABLE 1

| | Mixture of red ginseng | | |
|---|---|---|---|
| | extract/fraction (10 mg/ml) | | |
| | Beneficial | Harmful | Beneficial |
| | bacteria | bacteria | bacteria/harmful |
| | (%) | (%) | bacteria |
| Sodium propionate (5 mg/ml) | 194.0 | 6.0 | 32.34 |
| Plant extract (10 μl/ml) | 146.3 | 336.6 | 0.43 |
| Sodium benzoate (5 mg/ml) | 23.5 | 29.9 | 0.79 |
| Potassium sorbate (5 mg/ml) | 24.9 | 31.3 | 0.80 |

From the results of Table 1, in the case of the composition for improving intestinal microbial flora containing sodium propionate in the mixture of red ginseng extract/fraction, it was confirmed that the effect of selectively promoting the growth of beneficial bacteria versus harmful bacteria was excellent. On the other hand, in the case of the composition including the plant extract in the mixture of red ginseng extract/fraction, it was confirmed that the growth promoting effect of beneficial bacteria was excellent, but the growth of harmful bacteria was also greatly promoted, and accordingly, the effect of selectively promoting the growth of beneficial bacteria versus harmful bacteria was low.

### Example 3

3-1. Preparation of Composition for Improving Intestinal Microbial Flora

In the composition of the mixture of red ginseng extract/fraction and the sodium propionate, in order to confirm a selective growth promoting effect of beneficial bacteria versus harmful bacteria according to the content of sodium propionate, the mixtures were prepared by mixing 10 mg/ml of the mixture of red ginseng extract/fraction with sodium propionate at concentrations of 0, 1.25, 2.5, and 5 mg/ml, respectively.

In the case of further including a plant extract to the composition of the mixture of red ginseng extract/fraction and the sodium propionate, in order to confirm the selective growth promoting effect of beneficial bacteria versus harmful bacteria according to the content of sodium propionate, 10 mg/ml of the mixture of red ginseng extract/fraction was mixed with sodium propionate at concentrations of 0, 1.25, 2.5, and 5 mg/ml, respectively, and mixed with 10 μl/ml of the plant extract of Preparation Example 4 to prepare mixtures.

3-2. Confirmation of Selective Growth Promoting Effect of Beneficial Bacteria Versus Harmful Bacteria

In order to confirm whether each mixture selectively promoted the growth of beneficial bacteria versus harmful bacteria, each mixture was treated with physiological saline, and then the degrees of the growth of beneficial bacteria and harmful bacteria were measured in the same manner as in Example 1, and the increased amounts of the strains were calculated as a percentage compared to an untreated group and were shown in Table 2 below.

TABLE 2

| | | | | Mixture of red ginseng | |
|---|---|---|---|---|---|
| | Mixture of red ginseng | | | extract/fraction (10 mg/ml) + | |
| | extract/fraction (10 mg/ml) | | | Plant extract (10 μl/ml) | |
| | Beneficial | Harmful | Beneficial | Beneficial | Harmful |
| | bacteria | bacteria | bacteria/harmful | bacteria | bacteria |
| | (%) | (%) | bacteria | (%) | (%) |
| Sodium propionate (5 mg/ml) | 194.0 | 6.0 | 32.34 | 113.4 | −9.7 |
| Sodium propionate (2.5 mg/ml) | 179.1 | 7.5 | 23.96 | 110.4 | −23.9 |
| Sodium propionate (1.25 mg/ml) | 173.1 | 10.4 | 16.65 | 113.4 | −14.2 |
| Sodium propionate (0 mg/ml) | 168.7 | 332.8 | 0.51 | 146.3 | 336.6 |

From the results of Table 2, it was confirmed that in the case of the composition for improving intestinal microbial flora including the mixture of red ginseng extract/fraction and the sodium propionate, there was an effect of selectively promoting the growth of beneficial bacteria versus harmful bacteria. Particularly, it was confirmed that in the case of including 25 wt % (that is, in the case of a 2.5 mg/ml-concentration treated group) or more of sodium propionate based on the mixture of red ginseng extract/fraction, the selective promoting effect was increased. In addition, in the case of the composition for improving intestinal microbial flora further including the plant extract of Preparation Example 4 in the mixture of red ginseng extract/fraction and the sodium propionate, it was confirmed that as the growth of harmful bacteria was greatly reduced, the effect of selectively promoting the growth of beneficial bacteria was further maximized.

1. A composition for improving intestinal microbial flora, comprising:
a ginseng extract or a fraction thereof; and
sodium propionate.

2. The composition for improving the intestinal microbial flora of claim 1, comprising:
a ginseng extract;
a fraction of the ginseng extract; and
sodium propionate.

3. The composition for improving the intestinal microbial flora of claim 2, wherein the ginseng extract and the fraction of the ginseng extract are included in a weight ratio of 1:0.05 to 1:0.5.

4. The composition for improving the intestinal microbial flora of claim 2, wherein the fraction of the ginseng extract is a column-purified fraction of an aqueous alcohol solution of a ginseng alcohol extract.

5. The composition for improving the intestinal microbial flora of claim 1, wherein the weight ratio of the ginseng extract or the fraction thereof versus the sodium propionate is 4:1 to 1:4.

6. The composition for improving the intestinal microbial flora of claim 1, further comprising:
a plant extract or a fraction thereof.

7. The composition for improving the intestinal microbial flora of claim 6, wherein the weight ratio of the ginseng extract or the fraction thereof versus the plant extract or the fraction thereof is 6:1 to 1:6.

8. The composition for improving the intestinal microbial flora of claim 1, wherein the improving of the intestinal microbial flora is promoting the growth of intestinal beneficial bacteria, or inhibiting the growth of intestinal harmful bacteria.

9. The composition for improving the intestinal microbial flora of claim 8, wherein the intestinal beneficial bacteria include at least one selected from the group consisting of L. acidophilus, L. casei, L. gasseri, L. delbrueckii subsp. Bulgaricus, L. helveticus, L. fermentum, L. paracasei, L. plantarum, L. reuteri, L. rhamnosus, L. salivarius, L. delbrueckii subsp. lactis, L. lactis, L. lactis subsp. cremoris, L. lactis subsp. lactis biovar diacetylactis, L. lactis subsp. lactis, E. faecium, E. faecalis, S. thermophilus, L. mesenteroides, L. pseudomesenteroides, B. bifidum, B. infantis, B. breve, B. longum, B. animalis subsp. lactis, B. lactis, and B. coagulans.

10. The composition for improving the intestinal microbial flora of claim 8, wherein the intestinal harmful bacteria include at least one selected from the group consisting of E. coli, S. aureus, Acidaminobacter, Enterobacteriaceae, A. muciniphila, E. rectale, C. coccoides, C. lituseburense, C. histolyticum, C. perfringens, C. difficile, Veillonellaceae, Proteobacteria, Betaproteobacteria, P. aeruginosa, C. albicans, and B. vulgatus.

11. The composition for improving the intestinal microbial flora of claim 1, wherein the composition is a health functional food composition or a food additive composition.

12. A pharmaceutical composition for preventing, improving or treating intestinal inflammatory diseases or intestinal motility disorders, comprising:
a ginseng extract or a fraction thereof; and
sodium propionate.

13. The pharmaceutical composition of claim 12, comprising:
a ginseng extract;
a fraction of the ginseng extract; and
sodium propionate.

14. The pharmaceutical composition of claim 13, wherein the fraction of the ginseng extract is a column-purified fraction of an aqueous alcohol solution of a ginseng alcohol extract.

15. The pharmaceutical composition of claim 12, further comprising:
a plant extract or a fraction thereof.
